(19) United States
(12) Patent Application Publication
(10) Pub. No.: US 2017/0014314 A1
(43) Pub. Date: Jan. 19, 2017

(54) LIQUID COSMETIC COMPOSITION SUITABLE FOR CHANGING TEMPORARILY KERATINIC FIBERS COLOR - RELATED METHOD

(71) Applicant: BBR, Paris (FR)

(72) Inventor: Elena ALEXANDROVA, Croissy sur Seine, Paris (FR)

(21) Appl. No.: 15/118,016

(22) PCT Filed: Feb. 6, 2015
(86) PCT No.: PCT/FR2015/050292

§ 371 Date: Aug. 10, 2016

(30) Foreign Application Priority Data
Feb. 10, 2014 (FR) 1450991

(51) Int. Cl. A61K 8/04; A61K 8/33; A61K 8/34; A61K 8/41; A61K 8/19; A61K 8/25; A61K 8/29; A61Q 5/06; A61K 8/81

(52) U.S. Cl. CPC A61K 8/046; A61Q 5/065; A61K 8/33; A61K 8/345; A61K 8/8182; A61K 8/19; A61K 8/25; A61K 8/29; A61K 8/416; A61K 2800/222

(57) ABSTRACT

A cosmetic composition capable of temporarily modifying the colour of the hair, of the type containing: a cosmetically acceptable solvent which comprises at least one alcohol; at least one pigment chosen from metal oxides and hydroxides, in particular iron(II) oxides and hydroxides, iron(III) oxides and hydroxides, mixtures of at least two of said iron(II) or (III) oxides or hydroxides, in particular mixtures of at least two iron(II) and/or iron(III) oxides, mixture(s) of at least two iron(II) and/or iron(III) hydroxides, mixtures of at least one iron(II) or iron(III) oxide with at least one iron(II) or iron(III) hydroxide, and mixtures of at least one iron(II) or iron(III) oxide and/or at least one iron(II) or (III) hydroxide with titanium dioxide; and at least one polymer for fixing said pigment. The pigment is capable of giving the hair a natural colour and in particular of covering white hair and said composition is liquid. A process for temporarily dying the hair, with a view to giving it a natural colour, according to which a solution containing at least one pigment, one polymer fixing said pigment and one cosmetically acceptable solvent is sprayed onto said hair.

The present invention relates to a liquid cosmetic composition to temporarily change the color of keratin fibers, especially hair and a temporary hair coloring process.

The present invention relates more particularly to a cosmetic composition suitable for temporarily hiding the difference in hue between the hair roots and the rest of the hair by temporarily giving the roots area the same color as the rest of the hair; the aforementioned hue difference coming, for instance, from natural regrowth of hair that has been colored chemically.

When the hair is chemically colored or simply thinned by solar radiation, an unsightly bar appears at the hair roots because of hair growth. When hair is chemically colored to cover gray/white hair, we can observe, depending on the number and dispersion of white hair, a white bar unattractive at the roots.

Until now, to hide unsightly aforesaid area, it was necessary to use a chemical stain. The chemical coloring tends to damage the hair if one uses too frequently. Natural dyes and henna sheathing type do not offer a fairly wide range of colors and do not effectively cover gray hair.

There are temporary colorations which allow coloring the hair between two shampoos; the coloring is eliminated by the use of hot water and a surfactant.

Thus, document FR 2,679,771 A1 discloses gels for temporarily coloring the hair which contain as a pigment for coloring the hair, an insoluble pigment obtained by oxidative polymerization of indole derivatives.

This product is in the form of a gel, it can temporarily color the whole hair and cover gray hair but it remains difficult to use locally only on the roots. In addition, it takes some time to dry because it primarily contains water and its use therefore requires drying hair. Furthermore, the pigment used is of synthetic origin and may therefore cause reactions at the skin and scalp.

There are also styling gels that can cure the hair to give them the desired shape and which give the hair eccentric colors like green, gold or white. These gels cannot be used for temporary dyeing purposes to give the hair a natural color.

An object of the present invention is to provide a cosmetic composition for temporary coloring that can replace chemical coloring composition, particularly for hiding unsightly appearance of the hair roots having a color different from the hair length color, and in particular, for hiding white hair.

Another object of the present invention is to provide a cosmetic composition as aforesaid which is easy to use, particularly in the roots.

Another object of the present invention is to provide a cosmetic composition for temporary dyeing which does not cause any irritation, particularly in the skin and/or scalp.

Another object of the present invention is to provide a cosmetic composition for changing temporarily the color of keratin fiber, which can be easily removed so that, for example, not to interfere with possible chemical coloring or other chemical treatment of the fibers keratin, as a permanent waving or discoloration.

To achieve at least one of these purposes, the present invention provides a cosmetic composition capable of temporarily changing the hair color, containing:
a solvent acceptable in cosmetics and which comprises at least one alcohol,
at least one pigment selected from metal oxides and hydroxides, in particular iron oxides and hydroxides (II), oxides and hydroxides of iron (III), mixtures of at least two of said oxides or hydroxides of iron (II) or (III), in particular, mixtures of at least two iron (II) oxides and/or iron (III), mixture(s) of at least two hydroxides of iron (II) and/or iron (III), mixtures of at least one iron (II) or iron (III) oxide with at least one iron (II) hydroxide or iron (III) hydroxyde, and mixtures of at least one iron oxide (II) or one iron (III) oxide and/or at least one hydroxide of iron (II) or (III) with titanium dioxide; and
at least one fixing polymer of said pigment.

According to the invention, characteristically, said pigment is capable of imparting a natural color to the hair and in particular to cover white hair and said cosmetic composition is liquid.

Thus, the composition of the present invention makes it possible to cover the surface of the keratin fibers by the pigment or pigment mixture, the latter being in sufficient amount and having a suitable color to mask the color of hair in particular white hair. The composition is chemically inert with respect to keratin fibers, i.e. that there is no creation of any covalent bond between the keratin fibers and one or more components of the cosmetic composition according to the invention. The pigment is attached to the surface of the fiber by the binding polymer. The only links between the keratin fibers and the composition of the invention are low-energy bonds, Van der Waals type which are due in particular to fixing polymer. Accordingly, a temporary or fleeting coloring is obtained.

Advantageously, the composition of the invention is in the form of a spray able liquid solution or aerosol. Thereby, the composition of the invention can be applied easily and uniformly by spraying.

Thus, the composition may further comprise a liquid propellant, particularly dimethyl ether.

The composition of the invention may thus be in the form of a spray able solution, thanks in particular to a mechanical spray device. It may also include a propellant enabling to spray the composition of the invention through a pressurized spray bottle. The pigments are thus evenly distributed, forming a thin and homogeneous layer on the keratin fibers, especially the hair, particularly the hair roots. Thereby, the composition provides a uniform color to the keratinous fibers. The color imparted by the composition of the invention blends well with hair color without demarcation.

The solvent is not limited the invention. Advantageously, it is chosen from primary alcohols, secondary alcohols, alcohols, tertiary amines, water and mixtures of at least two of said solvents.

For example alcohol and mixture of alcohols include ethanol, propanol, ethylene glycol, denatured alcohol which is a mixture of ethanol and a denaturant, e.g., selected from diethyl phthalate, thiophene, diethyl ether, isopropyl alcohol, sodium salicylate, methyl salicylate, brucine, brucine sulfate and quassin.

The Applicant has demonstrated that a mixture of water and alcohol, in particular ethanol, optionally denatured ethanol containing predominantly (that is to say more than 50% by weight of alcohol) provides a good dispersion/dissolution of the pigment into the binding polymer, which allows obtaining a homogeneous solution and/or aerosol. Moreover, the composition of the invention comprising the above-mentioned alcohol water mixture as a solvent has the advantage of drying very quickly, solvent evaporation being fast.

Advantageously, the solvent is an alcohol+water mixture (s) that includes in weight more than 80% of alcohol (s) and in particular more than 90% of alcohol.

Applicants have demonstrated that such a solvent allows the solution to dry quickly without damaging the scalp or skin, or dry keratin fibers.

According to the invention, the pigment may be a powder insoluble in the solvent. The particle size of pigment powder and their shape are not limited according to the invention. For example, the average diameter of the pigment particles may be substantially equal to 1 μm or greater than 1 μm and substantially equal to or less than 8 μm. Finer the particle size is, the more the attachment is effective and rendering is natural. The particle size is to be adapted to the vaporization device used. Mixtures of the aforementioned pigments allow obtaining various shades from light blond to black, through the dark blond, light brown and dark brown. However, it is possible to envisage the use of other metal oxides, such as, for example, copper oxide or chromium oxide optionally in combination with one or more oxides or hydroxides of iron (II) or (Ill) to obtain a natural color.

The polymer fixation of the pigment is not limited according to the invention. It must adhere to the keratin fiber without chemical reaction. It can be, for example, selected from poly vinyl pyrrolidone, polyquaternium-4, PVM/MA copolymers, poly butylene terephthalate, polyethylene terephthalate, polyvinyl butyral (PVB), the VP/VA copolymers (copolymers ‘vinyl acetate and vinyl pyrrolidone), chitosan, sodium polyacrylate, dried xanthan gum, collagen, and mixtures of at least two of said film-forming agents.

Copolymers VP/VA are efficient to secure pigments, in particular iron oxides. These copolymers can be selected among VP/VA copolymers having a molecular weight of 735 and a weight ratio of VP/VA=70/30. These copolymers are liquids. Thereby, they enable obtaining a homogeneous colored film that covers the surface of the keratinous fibers. This film resists to rain, brushing; it does not stain fabrics that can come in contact therewith, for example, clothing, pillowcases, and hats or caps. The film leaves hair soft without hardening effect. It is possible to rub into the hair without removing the film and thus without getting your hands dirty. The colored film may remain for several days on the keratin fibers; it is not removed mechanically but a simple shampoo, preferably with warm water removes the film and the pigment: the keratin fibers then return to their original color.

Advantageously, the composition contains a percentage by weight of said alcohol(s) relative to the sum of the weight of said alcohol(s), the weight of said pigment(s) and the weight of said fixing polymer(s) which is substantially equal or greater than 75%, particularly substantially equal to or greater than 80% and more particularly substantially equal to 82%.

Advantageously, the composition according to the invention contains a weight of said fixing polymer(s) and said pigment(s) such as the weight ratio pigment(s)/fixing polymer(s) is substantially equal to or greater 2 and in particular substantially equal to or greater than 2.3 and more specifically substantially equal to or greater than 2.35 and in particular substantially equal to 2.4.

Advantageously, the cosmetic composition of the invention further contains at least one hygroscopic agent, preferably selected from among wetting agents, anti-caking agents selected from talc, corn starch, the double silicate of magnesium and aluminum, copolymers of adipic acid and neopentyl glycol, extracts of ivy, silicon dioxide, cyclo hepta siloxane, aluminum salt of the product of the reaction of octenyl succinic anhydride with starch, zinc stearate, zinc ricinoleate, magnesium silicate copolymers, dimethicone/methicone, microcrystalline cellulose, silicic acid, di and tri stearate, aluminum stearate, mixtures of at least two of said anti-caking agents and mixtures at least one wetting agent (moisturizing agent) with at least one anti-caking agent.

Preferably, talc and/or the product of the aluminum salt of the reaction of octenyl succinic anhydride with starch are used. They have more a function of anti-caking agent, avoiding the powder pigments to agglomerate. Agglomeration would decrease the uniformity of the colored film formed by the composition of the invention. The anti-caking agents tend to capture water from the environment in which they are located. In this case, the anti-caking agent will be contained in the film formed by the composition of the invention on the surface of keratinous fibers and coat the latter. This anti-caking agent will capture by absorption or adsorption of water in the air which will effectively give a thicker appearance to the keratin fiber. In the case of hair, the composition of the invention then allows to give body to the hair, in particular for thin hair.

The moisturizing agent (wetting agent) enables to inflate the hair and hydrate it so that it remains glossy. The moisturizing agent can also by itself help to give body to the keratin fiber by acting directly on the latter. The combination between a moisturizing agent and an anti-caking agent enables to obtain the two combined effects. Thereby, the composition provides to the keratin fiber a visible thicker appearance. In the case of the hair, hair has a greatly improved appearance. Hair is more brilliant and silky and has an increased volume. Moreover, the moisturizing agent prevents hair drying when the alcohol contained in composition evaporates.

The moisturizing agent may be selected, for example, from silk powder, propylene glycol, glycosyl trehalose, hydroxyethyl urea, silk amino acids, inositol and mixtures of at least two of said agents.

According to one embodiment, the weight of said moisturizing agent is such that the weight ratio of said moisturizing agent/fixing polymer(s) is substantially equal to or greater than 1.3 and substantially equal to or less than 1.7, and substantially equal to 1.5. This provides an efficient pigment fixation without drying the hair.

According to another embodiment, the weight of said moisturizing agent is such that the weight ratio of said moisturizing agent/pigment(s) is substantially equal to or greater than 0.5 and substantially equal to or less than 0.7 and in particular approximately equal to 0.6.

Advantageously, the weight of said hygroscopic agent is such that the weight ratio of said moisturizing agent/(sum of weights of said alcohol(s), said pigment(s) and said fixing polymer(s) is substantially greater or equal to 0.03 and substantially equal to or less than 0.04.

According to one embodiment, the weight percentage of said hygroscopic agent is substantially equal to or greater than or equal to 8% and substantially equal to or less than 20%, in particular, substantially equal to or greater than or equal to 18% and substantially equal to or less than 20%, especially substantially equal to or greater than or equal to 8% and substantially equal to or less than 9%. The weight percentage may be substantially equal or higher than 9.4 and less than or substantially equal to 9.6%.

Advantageously, the cosmetic composition of the invention may comprise, in addition, an antistatic agent, particularly an antistatic agent chosen from chloride dodecyl trimethyl ammonium, squalene and mixtures of said antistatic agents and/or UV-absorbing agent, particularly an agent UV absorber selected from benzyl salicylate, octyl salicylate, ethylhexyl methoxycinnamate, bark and bud extract of maritime pine, Galangal root extract, phenyl tetrabutyl hydroxy benzoate and benzophenones.

According to a particular embodiment which may be combined with the aforementioned ones, the weight percentage of said solvent or said solvent mixture is substantially equal to or greater than or equal to 60% and substantially equal to or less than 70%, especially substantially equal to 66%.

According to another embodiment which may be combined with any of the aforementioned embodiments, the weight percentage of pigment(s) is substantially equal to or greater than or equal to 9% and substantially equal to or less than 10%.

According to another embodiment which may be combined with any of the above embodiments, the weight percentage of said fixing agent is substantially equal to or greater than or equal to 3% and substantially equal to or less than 5%. It may advantageously be substantially equal to 4%.

Without precision, the weight percentages refer to the following report: weight of the considered component/(weight of the considered component+weight of solvent+weight of pigment (s)+weight of fixing polymer(s))×100.

The present invention also relates to an optionally pressurized spray bottle containing the cosmetic composition of the present invention and optionally a propellant. The present invention also relates to a temporary hair coloring process, in order to seek a natural color, according to which one vaporizes to said hair, a solution containing at least one pigment, a fixing polymer for fixing said pigment and solvent acceptable in cosmetics.

The pigment may be a mixture of metal oxides. Advantageously, the solution is that of the present invention.

According to a particular mode of implementation, said solution is vaporized only at the roots of the hair and in particular onto the roots which are visible on the surface of the skull.

The present invention also relates to a spray containing a propellant and the composition of the invention.

Definitions

The “cosmetically acceptable” terms mean in the sense of the present invention, any substance or preparation that can be placed in contact with the various external parts of the human body, including the skin and appendages or with the teeth and oral mucosa with a view exclusively or principally to cleaning them, perfuming them, changing their appearance and/or correcting body odors and/or protecting and/or maintain them in good condition.

The terms “keratin fibers” means especially the hair, the hair and lip down. The present invention is particularly intended for treating the hair.

The terms “temporary coloring” and derivative terms refer to a color that can be removed with hot water and possibly a surfactant such as a shampoo. We can also talk about fleeting coloring. The coloration obtained is not a transparent color. The visible color of the fiber is that one provided by the one or more pigment(s) without the natural color being visible there through.

The term “liquid” refers more particularly but not exclusively, a solution or a solid/liquid suspension which can be vaporized.

The term “aerosol” means a suspension of droplets and/or particles in a gaseous medium.

The term “pigment” refers to a compound insoluble in the solvent and can be present in powder form.

The term “iron oxides” means the iron oxides, iron hydroxides and iron oxides-hydroxides. iron oxides (II) and/or (III) are selected from iron (II) oxide FeO iron oxide (II, III) (Fe₃O₄ or FeO Fe₂O₃) and iron oxide (III) Fe₂O₃, in each of its four phases: iron hydroxides are iron (II) hydroxide Fe(OH)₂ green, iron oxyhydroxide (III) FeO(OH), red-brown and ferric hydroxide Fe (OH)₃, of dark brown color.

Among the iron oxides used, mention may be made of iron trioxide (CI 77491), iron oxide (CI 77492) and iron oxide tetra (CI 77 499). These three pigments associated enable a wide variety of shades ranging from blond to black.

The terms “natural colors” are the hues and shades that coat the hair naturally, without any coloring. It's black, red, brown, auburn, blond and brown and all shades of these colors. According to the invention, white color which has to be hidden by the composition of the invention is not considered as a natural color in the sense of the present invention.

The invention, its features and the various benefits it provides will be explained in more details on the basis of the following examples.

### EXAMPLES

All examples below relate to compositions in the form of a spray able solution containing a propellant.

### Example 1

### Composition for Imparting a Golden Blonde Color, Especially to White Hair

TABLE I

| Compound | Weight percentage |
|---|---|
| Dimethyl ether | 80 |
| Denatured alcohol | 12.22 |
| aluminum salt of the product | 2.112 |
| of the reaction of anhydride | |
| octenyl succinic with starch | |
| Propylene glycol | 1.20000 |
| water | 1.08640 |
| Copolymer VP/VA | 0.80000 |
| Iron oxides (CI 77492) | 0.67200 |
| Iron oxides (CI 77499) | 0.64000 |
| talc | 0.52000 |
| Iron oxides (CI 77491) | 0.28800 |
| Titanium dioxide (CI77891) | 0.28000 |
| Fragrances(linalool, geraniol) | 0.18000 |
| dodecyltrimethyl ammonium chloride | 0.00160 |

### Example 2

### Composition for Imparting Dark Blonde Color to Hair Especially White Hair

TABLE II

| Compound | Weight percentage |
|---|---|
| Dimethyl ether | 80 |
| Denatured alcohol | 12.22 |
| aluminum salt of the product | 2.112 |
| of the reaction of anhydride | |
| octenyl succinic with starch | |
| Propylene glycol | 1.20000 |
| water | 1.08640 |
| Copolymer VP/VA | 0.80000 |
| Iron Oxides (CI 77492) | 0.46400 |
| Iron oxides (CI 77499) | 0.76800 |
| talc | 0.52000 |
| Iron oxides (CI 77491) | 0.36800 |
| Titanium dioxide (CI77891) | 0.28000 |
| Fragrances(linalool, geraniol) | 0.18000 |
| Dodecyltrimethyl ammonium chloride | 0.00160 |

### Example 3

### Composition for Imparting a Light Brown Coloration to Hair, Especially to White Hair

TABLE III

| Compound | Weight percentage |
|---|---|
| Dimethyl ether | 80 |
| Denatured alcohol | 12.22 |
| aluminum salt of the product | 2.112 |
| of the reaction of anhydride | |
| octenyl succinic with starch | |
| Propylene glycol | 1.20000 |
| water | 1.08640 |
| Copolymer VP/VA | 0.80000 |
| Iron oxides (CI 77492) | 0.46400 |
| Iron oxides (CI 77499) | 1.08800 |
| talc | 0.48000 |
| Iron oxides (CI 77491) | 0.36800 |
| Titanium dioxide (CI77891) | none |
| Fragrances (linalool, geraniol) | 0.18000 |
| dodecyltrimethyl ammonium chloride | 0.00160 |

### Example 4

### Composition for Imparting Dark Brown Color to White Hair

TABLE IV

| Compound | Weight percentage |
|---|---|
| Dimethyl ether | 80 |
| Denatured alcohol | 12.22 |
| aluminum salt of the product | 2.112 |
| of the reaction of anhydride | |
| octenyl succinic with starch | |
| Propylene glycol | 1.20000 |
| water | 1.08640 |
| Copolymer VP/VA | 0.80000 |
| Iron oxides (CI 77492) | none |
| Iron oxides (CI 77499) | 1.92000 |
| talc | 0.4800 |
| Iron oxides (CI 77491) | none |
| Titanium dioxide (CI77891) | nonet |
| Fragrances(linalool, geraniol) | 0.18000 |
| dodecyltrimethyl ammonium chloride | 0.00160 |

Example of Use of the Composition According to the Invention

The composition used in this example is a composition in a spray bottle containing propellant gas.

Stir the bottle which further contains a glass ball to ensure perfect homogeneity of the composition. Maintain vertically said bottle, head up and vaporize the composition of the invention at 20 cm from hair—after drying and/or brushing-, where the roots are visible, depending on the hairstyle. One proceeds by pressing and moving the bottle according to a hair line. On a spike, a couple of quick pressure is enough. If the hair is raised, pulled back, forming a bun or ponytail, small sprays is done along the edge of the hair, all around the head. Allow drying (drying time: 1 minute).

The composition according to the invention can be used to cover gray hair but also a fun way on a few strands to create contrast, bring the matter to color, play darker roots to highlight blonde spikes, get a “tie and dye effect.

Evaluation of the Skin Compatibility of the Composition According to the Invention After Single Application Under Semi-Occlusive Dressing for 48 Hours (Test Carried Out on 11 Adult Volunteers)

Irritation potential of the compositions listed in Tables I to IV above was determined on 10 volunteers. A single application of 160 μL is carried out on the anterolateral surface of the arm. The skin zone is kept in contact with the composition for 48 hours using a semi-occlusive bandage.

The 10 volunteers, male or female, aged 18 to 65 have normal skin and skin free of evolving dermatological lesion. The clinical scoring is carried out half an hour after patch removal and takes into account erythema, edema, papules, vesicles and bubbles. Depending on their intensity, scoring ranges from 0 to 3. The sum of the scores divided by the number of subjects sets the index of average irritation (I.I.M) which classifies the test element according to the schedule below:

I.I.M.<0.20 Non irritating 0.20<I.I.M.<0.50 Slightly irritating 0.50<I.I.M.<2 Moderately irritating 2<I.I.M.<3 Very irritating

Results: The average irritation index (IIM) of the composition according to the invention obtained is 0, 0 which proves that the composition of the invention proves non-irritating even in direct contact with the skin for 48 hours.

Evaluation of the Drying Time of the Composition According to the Invention, the Time and Water Resistance of the Coloring

The following tests were performed on 20 women aged 18 to 70 with long or medium length hair, colored dark brown, brown or black with white or blonde roots.

According to the hair color, the composition of the invention described in one of the above tables was sprayed at a distance of 20 cm, on the roots visible at the parting of hair.

a) Evaluation of the Long-Lasting Effect, i.e. 8 Hours After Application of the Composition According to the Invention.

Clinical assistant and volunteers evaluated the coloring of the product of the invention, obtained just after the drying of the composition, then 8 hours later. The coloring rating scale is graduated from 0 (no effect covering) to 9 (total coverage of the color of the roots)

The clinical assistant believes that right after applying the product of the invention, coloring rates average of 8.95 and 4.86, 8 hours later. The standard deviation is 0.22, respectively, and 1, 42.

Volunteers estimate that coloring (i.e. covering hair) appears immediately after application of the product is 7.48 and it drops to 4.24, 8 hours later. The standard deviation is respectively 1, 33 and 1, 45 for the two above average values. The clinical assistant esteem of the persistence % is equal to 54.26 and 56.69 to the volunteers.

The product according to the invention has good stability over time, is well tolerated and can be applied daily or twice daily.

b) Drying Time

Clinical assistant and volunteers evaluate the drying time by observing the disappearance of the effect of wet hair after applying the product of the invention. The drying time measured by the clinical assistant is 1 min 14 s. The drying time assessed by the volunteers is of 57 s. The composition according to the invention dries very quickly.

c) Water Resistance

The clinical assistant evaluates the water resistance by evaluating the coloring (covering effect) with the same scale as above immediately after application and after water spraying 15 minutes after application of the product according to the invention.

The clinical assistant believes that right after applying the product of the invention, the medium covering effect is 8.75 and 6.55, after water spraying 15 minutes later. The standard deviation is respectively 0.44 and 1, 15. The clinical assistant considers that the persistence % is equal to 74.86. The product of the invention thus is resistant to water, rain even shortly after application.

1. A Cosmetic composition suitable for temporarily changing hair color, said composition containing:
a solvent acceptable in cosmetics and which comprises at least one alcohol,
at least one pigment selected from metal oxides and hydroxides, in particular iron oxides and hydroxides (II), oxides and hydroxides of iron (III), mixtures of at least two of said oxides or hydroxides of iron (II) or (III), in particular, mixtures of at least two iron (II) oxides and/or iron (III) oxides, mixture(s) of at least two hydroxides of iron (II) and/or iron (III), mixtures of at least one iron (II) oxide or one iron (III) oxide with at least one iron (II) hydroxide or iron (III) hydroxide, and mixtures of at least one iron oxide (II) or iron (III) and/or at least one hydroxide of iron (II) or iron (III) with titanium dioxide; and
at least one fixing polymer of said pigment,
wherein said pigment is capable of imparting a natural color to the hair and in particular to cover gray hair, and in that said composition is liquid.

2. The composition according to claim 1, wherein it further comprises a liquid propellant, in particular dimethyl ether.

3. The composition of claim 1, wherein said cosmetically acceptable solvent is chosen from primary alcohols, secondary alcohols, alcohols, tertiary amines, mixtures of at least two of said alcohols and mixtures of water and at least one alcohol chosen from primary alcohols, secondary alcohols and tertiary alcohols.

4. The composition according to claim 1, wherein said solvent is a mixture of water and alcohol(s) and comprises at least 80% alcohol (s) and in particular at least 90% alcohol(s).

5. The composition according to claim 1, wherein said fixing polymer is selected among polyvinyl pyrrolidone, polyquaternium-4, PVM/MA copolymers, polybutylene terephthalate, polyethylene terephthalate, polyvinyl butyral (PVB), VPA copolymers/A (vinyl acetate copolymers and vinyl pyrrolidone, chitosan, sodium polyacrylate, dried xanthan gum, collagen, and mixtures of at least two of said fixing polymers.

6. The composition according to claim 1, wherein the composition contains a weight percentage of said alcohol(s) with respect to the sum of the weights of said alcohol(s), said pigment(s) and said fixing polymer(s) which is substantially equal or greater than 75%, especially substantially at or above 80% and in particular substantially equal to 82%.

7. The Composition according to claim 1, wherein the composition contains a weight of fixing polymer(s) and a weight of pigment(s) such as the weight ratio pigment(s)/polymer(s) is substantially equal to or greater than 2 and in particular substantially equal to or greater than 2.3 and mere specifically substantially equal to or greater than 2.35 and in particular substantially equal to 2.4.

8. The composition according to claim 1, wherein the composition additionally contains at least one hygroscopic agent, preferably selected from among moisturizing agents, anti-caking agents selected from talc, corn starch, silicate dual magnesium and aluminum, copolymers of adipic acid and neopentyl glycol, extracts of ivy, silicon dioxide, cyclohepta siloxane, aluminum salt of the product of the reaction of octenyl succinic anhydride with starch, zinc stearate, zinc ricinoleate, magnesium silicate copolymers, dimethicone/methicone, microcrystalline cellulose, silicic acid, di and tri stearate, aluminum stearate, mixtures of at least two of said anti-caking agents and mixtures of at least one moisturizing agent with at least one anticaking agent.

9. The composition according to claim 1, further comprising at least one moisturizing agent selected from silk powder agent, propylene glycol, glycosyl trehalose, hydroxyethyl urea, silk amino acids, inositol and mixtures of at least two of said moisturizing agents.

10. The composition according to claim 8, wherein the weight of said moisturizing agent is such that the weight ratio moisturizing agent/fixing polymer(s) is substantially equal to or greater than 1.3 and substantially equal to or less than 1.7, and substantially equal to 1.5.

11. The composition according to claim 8, wherein the weight of said moisturizing agent is such that the weight ratio moisturizing agent/pigment(s) is substantially equal to or greater than 0.5 and substantially equal to or less to 0.7 and substantially 0.6.

12. The composition according to claim 6, wherein the weight of said hygroscopic agent is such that the weight ratio hygroscopic agent/(sum of weights of said alcohol(s), said pigment(s) and said fixing polymer(s)) is substantially greater than or equal to 0.03 and substantially equal to or less than 0.04.

13. The composition according to claim 1 wherein the composition further comprises an antistatic agent, particularly an antistatic agent selected from the dodecyl trimethyl ammonium chloride, squalene and mixtures of said antistatic agents and/or agent UV-absorbing, in particular a UV absorber selected from benzyl salicylate, octyl salicylate, ethylhexyl methoxycinnamate, bark and dud extracts of maritime pine, galangal root extract, tetrabutyl phenyl hydroxybenzoate and benzophenones.

14. A process for temporarily coloring hair, in order to confer a natural color thereto, wherein a solution containing at least one pigment, fixing polymer for fixing said pigment and a solvent acceptable in cosmetics is sprayed to said hair.

15. A process according to claim 14, wherein said solution is as defined in claim 1.

16. Method according to claim 14, wherein said solution is only spayed onto the hair roots and in particular onto the roots which are visible on the surface of the scalp.

17. A spray containing a propellant and a composition according to claim 1.
